(19) United States
(12) Patent Application Publication
(10) Pub. No.: US 2024/0261276 A1
(43) Pub. Date: Aug. 8, 2024

(54) NITROXOLINE FOR USE IN THE TREATMENT OF CUTANEOUS NEUROFIBROMA

(71) Applicant: HEALX LTD, Cambridge, Cambridgeshire (GB)

(72) Inventor: David BROWN, Cambridge, Cambridgeshire (GB)

(21) Appl. No.: 18/567,829

(22) PCT Filed: Jun. 9, 2022
(86) PCT No.: PCT/GB2022/051442

§ 371 Date: Dec. 7, 2023

(30) Foreign Application Priority Data
Jun. 9, 2021 (GB) 2108224.3

(51) Int. Cl. A61K 31/47; A61K 31/4184; A61P 35/00

(52) U.S. Cl. CPC A61K 31/47; A61K 31/4184; A61P 35/00

(57) ABSTRACT

The present invention relates to a composition comprising nitroxoline, or a pharmaceutically acceptable salt thereof, for use in the treatment or prevention of a cutaneous neurofibroma.

### FIELD OF THE INVENTION

This invention relates to new uses of nitroxoline.

### Background of the Invention

A neurofibroma is a benign nerve-sheath tumour in the peripheral nervous system. In 90% of cases, they are found as stand-alone tumours, while the remainder are found in persons with neurofibromatosis type I (NF1), an autosomal-dominant genetically inherited disease. Neurofibromas can result in a range of symptoms from physical disfiguration and pain to cognitive disability and can transform into malignant tumours.

Cutaneous (or dermal) neurofibromas (cNFs) originate in nerves in the skin and are typically associated with a single peripheral nerve. All neurofibromas are composed of a mixture of NF1 mutant Schwann cells (SCs) with other nerve fiber elements, such as axons, fibroblasts, mastocytes, macrophages and endothelial cells. Three kinds of cNFs are distinguished: 1) Discrete cNFs; sessile or pedunculated masses on the skin, which are fleshy and non-tender, and can vary in size, 2) Discrete sub-cNFs; lie below and look like bumps on the skin, which can sometimes be tender, and 3) Deep nodular neurofibromas; Involving tissues and organs underneath the dermis, but otherwise resembling cutaneous and subcutaneous neurofibromas.

NF1 is caused by germline mutations of the NF1 tumor suppressor gene, which encodes the protein neurofibromin. Neurofibromin functions as a GTPase-activating (GAP) protein and inactivates the intracellular signal transduction protein Ras by converting the active GTP-bound form into its inactive GDP-bound form. This in turn leads to the downregulation of Ras activity. Loss of neurofibromin activity increases Ras activity, which in turn promotes the transcription of a number of genes required for cell growth and proliferation. Cutaneous neurofibromas appear in at least 99% of patients with NF1 and originate from Schwann cell lineage in the dermis.

cNFs typically appear at puberty and tend to increase in number throughout life, so that they may reach thousands. Although of benign character, these tumors are disfiguring and often itching and painful, thus significantly affecting quality of life. There have also been considerable efforts to identify pharmacological targets to treat cutaneous neurofibromas. In particular cutaneous neurofibromas have been a frequent target of repurposing efforts as well as repositioning of drugs in development. Many different standards and methods have been applied to this task. In many cases, repurposing candidates have been identified based primarily on clinical pattern matching, while in others basic disease mechanisms have been studied extensively to identify therapeutic targets, followed by thorough preclinical validation.

Currently, there are no drugs approved for cNFs. Physical removal remains the most effective method for treating cNF: surgical excision or destruction by CO₂ laser, electrodessication, and ablation. Challenges facing removal include tumor regrowth from incomplete excision, significant scarring, and cost burden. There are on-going pilot phase II trial studies on selumetinib in treating patients with neurofibromatosis type 1 and cutaneous neurofibromas. However, it is unclear currently how successful this will be. Selumetinib is a selective inhibitor of mitogen-activated protein kinase kinase (MAPK kinase, MEK, MAP2K, and MAPKK) and has the systemic name 6-(4-bromo-2-chloroanilino)-7-fluoro-N-(2-hydroxyethoxy)-3-methylbenzimidazole-5-carboxamide.

Overall, efforts to treat cutaneous neurofibromas have led to some exciting possibilities, but no definitive successes, despite much effort. This has highlighted the need for new therapies.

Nitroxoline is used in humans as an antibiotic, it is not widely used but has been on the market since the 1960s. It is used in the treatment or prevention of biofilm infections, such as urinary tract infections. It is particularly effective at disrupting biofilms and it is the metal cation chelation property that is believed to be responsible for this action. Nitroxoline is metabolised in the liver to the corresponding sulphate and glucuronide metabolites. There is evidence that the metabolites both share the antimicrobial activity. It has also been used in anticancer settings via antiproliferative action. Nitroxoline has the systematic name 5-nitroquinolin-8-ol.

### SUMMARY OF THE INVENTION

The present invention is a composition comprising nitroxoline, or a pharmaceutically acceptable salt thereof, for use in the treatment or prevention of a cutaneous neurofibroma. As will be evident from the in vitro data presented below, nitroxoline is effective in treating and preventing a cutaneous neurofibroma.

A first aspect of the invention is a composition comprising nitroxoline, or a pharmaceutically acceptable salt thereof, for use in the treatment or prevention of a cutaneous neurofibroma.

A second aspect of the invention is use of nitroxoline, or a pharmaceutically acceptable salt thereof, for the manufacture of a medicament for use in the treatment or prevention of a cutaneous neurofibroma.

A third aspect of the invention provides a method of treating or preventing a cutaneous neurofibroma comprising administering the patient with a composition comprising nitroxoline or a pharmaceutically acceptable salt thereof.

### DESCRIPTION OF THE FIGURES

FIG. 1 shows the preparation and passage of the floating sphere cultures for the in vitro study.

FIG. 2 shows the dose response effect of nitroxoline in the proliferation of Tom+NF1+/− and Tom+NF1−/− cells.

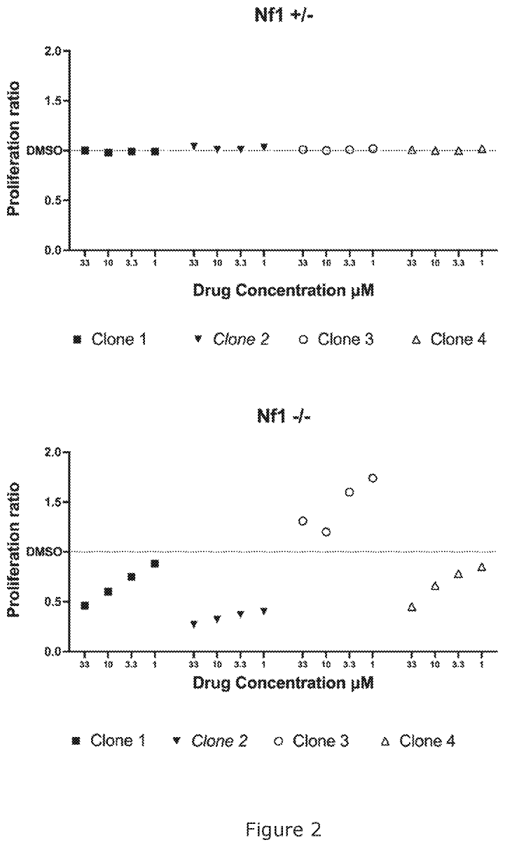

FIG. 3 shows the dose response effect of nitroxoline inducing cell death of Tom+NF1+/− and Tom+NF1−/− cells.

FIG. 4 shows fluorescent cNF tumour cells in the mouse skin using low power (5×) magnification. A-D: Representative images of Tom+ cells in two mice after one month of vehicle treatment; E-H: Representative images of Tom+ cells in two mice after one month of nitroxoline treatment.

### DETAILED DESCRIOTION

In the present invention, and as demonstrated by the below in vitro and in vivo data, nitroxoline inhibits cell proliferation and increases apoptosis in tumor cells isolated from cutaneous neurofibromas derived from the NF1−/− mouse model, and is therefore an effective treatment of a cutaneous neurofibroma. Preferably, nitroxoline is used for the treatment or prevention of a cutaneous neurofibroma, wherein the subject has neurofibromatosis type I.

By the term “treatment” or “treating” as used herein, we refer to therapeutic (curative) treatment and/or amelioration treatment (improvement in a patient's condition), which includes reducing the size of a cutaneous neurofibroma or the number of cNFs. By the term “prevention” or “preventing” as used herein, we refer to “prophylactic” treatment to prevent cNFs forming. This includes administering the compositions of the invention to a patient that has NF1 but a cutaneous neurofibroma has not developed or to a patient that has a cNF but the aim is to prevent more from developing.

“Patient” and “subject” are used interchangeably and refer to the subject that is to be administered the nitroxoline. Preferably the subject is a human. Suitably the subject has neurofibromatosis type I. In one embodiment the subject is an adult. An “adult” is a person of 18 years of age or older. In another embodiment the subject is undergoing puberty, such as the subject is between 8 and 18 years old.

In one embodiment, nitroxoline is used for the treatment or prevention of a cutaneous neurofibroma, wherein the patient has had or is going to have surgery to remove some or all of the cutaneous neurofibroma. This may be particularly advantageous if the cutaneous neurofibroma is large and/or expands across tissue boundaries, so it is difficult to remove it all by surgery and/or a quick removal of at least some of it is desired/beneficial.

The term “surgery” has its normal meaning in the art. Surgery is an invasive technique with the fundamental principle of physical intervention on organs/organ systems/tissues for diagnostic or therapeutic reasons.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

The present invention is directed to a composition comprising nitroxoline, or a pharmaceutically acceptable salt thereof, for use in the treatment or prevention of a cutaneous neurofibroma.

In an alternative embodiment, the present invention is directed to a composition comprising nitroxoline, or a pharmaceutically acceptable salt thereof, for use in the treatment or prevention of a cutaneous neurofibroma, wherein nitroxoline is the only active agent in the composition. By only active agent it is meant that the composition does not contain other components which may be used in the treatment or prevention of a cutaneous neurofibroma. In an alternative embodiment, the composition further comprises a second active agent for treating cutaneous neurofibroma, preferably wherein the second active agent is selumetinib, or a pharmaceutically acceptable salt thereof.

In an alternative embodiment, the present invention is directed to a composition comprising nitroxoline, or a pharmaceutically acceptable salt thereof, for use in combination with a second composition comprising selumetinib, or a pharmaceutically acceptable salt thereof, wherein the two compositions are administered to the subject simultaneously, separately or sequentially.

As used herein, “separate” administration means that the drugs are administered as part of the same overall dosage regimen (which could comprise a number of days), but preferably on the same day. As used herein “simultaneously” means that the drugs are to be taken together or formulated as a single composition. As used herein, “sequentially” means that the drugs are administered at about the same time, and preferably within about 1 hour of each other. Preferably, the drugs are administered simultaneously i.e. taken together or formulated as a single composition. Most preferably, they are formulated as a single composition.

The compositions of the invention may contain a pharmaceutically acceptable carrier. By “pharmaceutically acceptable carrier” is meant any diluent or excipient, such as fillers or binders, that is compatible with the other ingredients of the composition, and which is not deleterious to the recipient. The pharmaceutically acceptable carrier can be selected on the basis of the desired route of administration, in accordance with standard pharmaceutical practices.

In the present invention, the composition may be administered in a variety of dosage forms. In one embodiment, the composition may be formulated in a format suitable for oral, rectal, parenteral, intranasal or transdermal administration or administration by inhalation or by suppository.

The composition may be administered orally, for example as tablets, troches, lozenges, aqueous or oily suspensions, dispersible powders or granules. Preferably, the composition is formulated such that it is suitable for oral administration, for example tablets and capsules. Tablets and capsules may be prepared with binding agents, for example, syrup, acacia, gelatin, sorbitol, tragacanth, celluloses or polyvinylpyrrolidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents and surfactants such as lecithin, or acacia; vegetable oils such as almond oil, coconut oil, cod liver oil, or peanut oil; preservatives such as butylated hydroxyanisole (BHA) and butylated hydroxytoluene (BHT). Liquid compositions may be encapsulated in, for example, gelatin to provide a unit dosage form.

The composition may also be administered parenterally, whether subcutaneously, intravenously, intramuscularly, intrasternally, transdermally or by infusion techniques. The composition may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed.

The present invention also provides an inhalation device containing the composition of the present invention. Typically said device is a metered dose inhaler (MDI), which contains a pharmaceutically acceptable chemical propellant to push the medication out of the inhaler.

The composition may also be administered by intranasal administration. The nasal cavity's highly permeable tissue is very receptive to medication and absorbs it quickly and efficiently. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing the composition according to the present invention.

The composition may also be administered by transdermal administration. For topical delivery, transdermal and transmucosal patches, creams, ointments, jellies, solutions or suspensions, or micro needling may be employed. The present invention therefore also provides a transdermal patch containing the composition.

The composition may also be administered by sublingual administration. The present invention therefore also provides a sub-lingual tablet comprising the composition.

The composition may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In an embodiment of the invention, the composition is administered in an effective amount to treat or prevent a cutaneous neurofibroma. An effective dose will be apparent to one skilled in the art, and is dependent on a number of factors including age, sex, weigh, which the medical practitioner will be capable of determining.

In a preferred embodiment, the composition comprises 30 mg to 600 mg, preferably 50 mg to 500 mg, more preferably 100 mg to 400 mg, yet more preferably 150 mg to 350 mg, most preferably 200 mg to 300 mg nitroxoline.

The composition may be administered once a day, twice a day, three times a day or four times a day.

In an embodiment of the invention, the composition is administered at least once a day. Preferably it is administered as a single daily dose. Preferably the single daily dose is 90 mg to 1800 mg, preferably 150 mg to 1500 mg, more preferably 300 mg to 1200 mg, yet more preferably 450 mg to 1050 mg, most preferably 600 mg to 900 mg of nitroxoline.

In an embodiment of the invention, the composition is administered twice daily. Preferably each dose is 45 mg to 900 mg, preferably 75 mg to 750 mg, more preferably 150 mg to 600 mg, yet more preferably 225 mg to 525 mg, most preferably 300 mg to 450 mg of nitroxoline.

In an embodiment of the invention, the composition is administered three times daily. Preferably each dose is 30 mg to 600 mg, preferably 50 mg to 500 mg, more preferably 100 mg to 400 mg, yet more preferably 150 mg to 350 mg, most preferably 200 mg to 300 mg of nitroxoline.

In an embodiment of the invention, the composition is administered four times daily. Preferably each dose is 15 mg to 500 mg, preferably 50 mg to 400 mg, more preferably 100 mg to 300 mg, yet more preferably 125 mg to 225 mg, most preferably 150 mg to 200 mg of nitroxoline.

Preferably, the dosage regime is such that the total daily dosage of nitroxoline does not exceed 1500 mg.

Suitably the effective dose of nitroxoline results in a concentration of 1 to 75 μM, preferably 5 to 50 μM, more preferably 10 to 40 μM in cells.

Suitably the composition comprising nitroxoline and the second composition comprising the second active agent, preferably selumetinib, are a single daily dose. Suitably the two compositions are administered simultaneously i.e. nitroxoline and selumetinib are taken together. The compositions may also be administered sequentially i.e. at about the same time, and preferably within about 1 hour of each other.

In the embodiments wherein the composition comprises selumetinib or the composition is for use in combination with a second composition comprising selumetinib, suitably the compositions comprising selumetinib comprise between 1 mg and 75 mg of selumetinib, preferably between 5 mg to 50 mg of selumetinib, more preferably between 10 mg to 35 mg of selumetinib, most preferably between 15 mg to 30 mg of selumetinib.

Suitably the effective dose of selumetinib administered to the subject is between 1 mg/m² and 75 mg/m² of selumetinib, preferably between 5 mg/m² to 50 mg/m² of selumetinib, more preferably between 10 mg/m² to 35 mg/m² of selumetinib, most preferably between 15 mg/m² to 30 mg/m² of selumetinib.

In order to treat or prevent a cutaneous neurofibroma, the composition comprising nitroxoline is used in a chronic dosage regime i.e. chronic, long-term treatment. Suitably the regime lasts for at least one month, suitably at least two months, such as at least three months.

The present invention also relates to a kit comprising: (i) at least one dose of nitroxoline, or a pharmaceutically acceptable salt thereof; and optionally (ii) at least one dose of selumetinib, or a pharmaceutically acceptable salt thereof, for simultaneous, separate or sequential use in the treatment or prevention of a cutaneous neurofibroma.

The present invention also relates to use of nitroxoline, or a pharmaceutically acceptable salt thereof, for the manufacture of a medicament for use in the treatment or prevention of a cutaneous neurofibroma. This embodiment of the invention may have any of the preferred features described above.

The present invention also relates to a method of treating or preventing a cutaneous neurofibroma comprising administering the patient with a composition comprising nitroxoline or a pharmaceutically acceptable salt thereof. This embodiment of the invention may have any of the preferred features described above. The method of administration may be according to any of the routes described above.

For the avoidance of doubt, the present invention also embraces prodrugs which react in vivo to give a compound of the present invention.

### Experimental Section

### Example 1—In Vitro Drug Testing Utilizing Tomato (Tom)+ Stem-Like Glial Cells at the Origin of cNFs

This study uses a Nf1-KO mouse strain(Prss56Cre/+, R26tdTom/+, NF1fl/fl) developing cutaneous neurofibromas (cNFs) that faithfully recapitulates human disease (Radomska et a., 2019). In this model, simultaneous bi-allelic loss of Nf1 and expression of Tomato fluorescent reporters were targeted into glial-stem like cells at the origin of cNFs. The assay was performed ex vivo using non-adherent cells in which Tomato expressing Nf1−/− and Nf1+/− stem-like cells from skin can be amplified, and their properties further characterized. While in this experimental condition, all differentiated cells die rapidly (24 h), glial-stem like cells at the origin of cNFs survive, proliferate and form neurospheres (multicellular compact structures) that can be propagated for long periods, hence preserving their in vivo properties. In this system, Nf1−/− cells express high levels of p-ERK (as expected due to the permanent activation of the RAS pathway), proliferate much faster compared to controls and preserve the capacity to reform spheres after dissociation and re-plating. Moreover, we observed that the incubation of Nf1−/− and Nf1+/− cells with Selumetinib (inhibitor of MEK1/2) decreases proliferative activity and promotes death of mutant without affecting Nf1+/− cells supporting the robustness of the in vitro system to perform drug screening studies.

This model was used to assess the effect of Nitroxoline on proliferation and cell death of Nf1−/− tumor cells.

### Model Characterization

This in vitro culture system uses Tomato (Tom)+ stem-like glial cells at the origin of cNFs that can be amplified and propagated for the long periods. Briefly, skin from young mutant (NF1−/−) and control (NF1+/−) mice was dissected, dissociated and cell suspension incubated in the non-adherent condition with the presence of two mitogens, FGF and EGF. In such conditions only stem-like cells survive, proliferate and form compact multicellular floating structures called neurospheres. Since, in addition to the Tom+ glial stem-like cells, skin contains various other types of stem cells (Tom−), the neurospheres are composed of the intermingled Tom+ and Tom− cells. While in the mutant condition, neurospheres contain Tom+, Nf1−/− and Tom−, Nf1+/+ cells, in the Nf1+/− control condition neurospheres are composed of Tom+, Nf1+/− and Tom−, Nf1+/+ cells. In this study we have analysed the drug effect in Tom+ from NF1−/− and NfF1+/− mice.

### Experimental Design

### Cell Preparation, Clonal Expansion and Drug Treatment

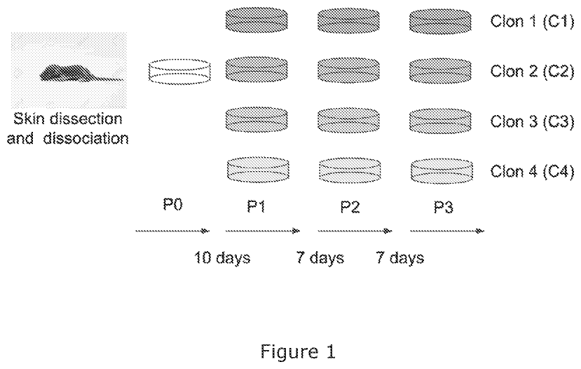

In the present study, floating sphere culture of Nf1+/−(Prss56Cre, R26Tom, Nf1flox/+) and Nf1−/− (Prss56Cre, R26Tom, Nf1flox/flox) glial cells isolated from newborn mouse skin were used. Then they are plated and treated and analysed independently. With this intent, Nf1+/− and Nf1−/− spheres were amplified until passage 3 (1 passage corresponds to 7 days of culture). At the end of PO (10 days), cells were split into 4 identical batches (clones 1-4), and were amplified to P3 (FIG. 1).

At the end of P3, spheres from each clone were dissociated and cells transferred into two 12 well plates. 6 different concentrations (100 μm, 33 μm, 10 μm, 3 μm, 1 μm and 0 μm) of drug were added and cells were incubated for 72 h. An additional 12 well plate containing cells with DMSO (vehicle used for the drug resuspension) was used as a reference for the normalization of results. After 72 h analyses was performed the on the non-fixed cells: proliferative and cell death assay.

### Results

### Proliferative Activity

For the analysis of proliferative activity, a cell tracker lipophilic dye (CellTrace Proliferation Kit, Thermo Fishers) was used. The cell tacker was added at the same time as the drugs. Cells were incubated for 72 h and proliferative activity was measured by cytometry analysis using LSR Fortessa X20 (BD Biosciences). This method measures a mean fluorescence intensity (MIF) for each condition. The condition with DMSO was used as a reference.

$$Ratio : \% Dead Cells ( treatment ) / \% Dead Cells ( DMSO ) =$$

$$If ratio > 1 : more dead cell compared to DMSO$$

$$If ratio > 1 : more dead cell compared to DMSO$$

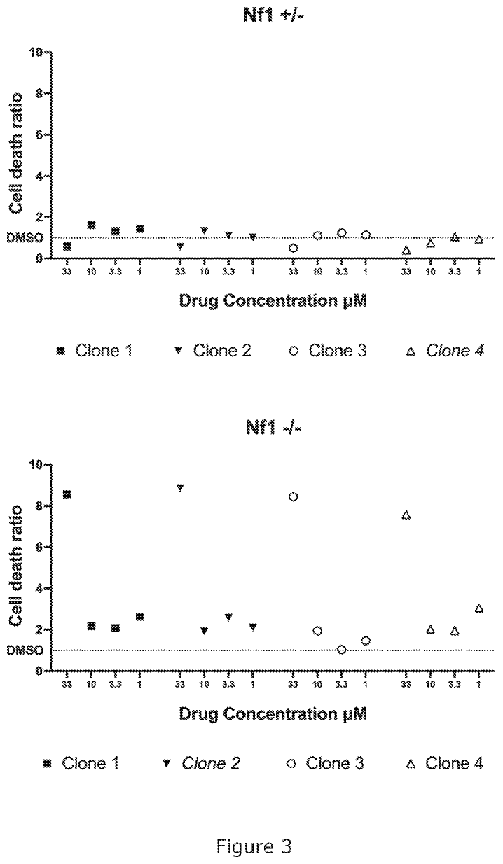

The highest concentration of nitroxoline tested in the assay, 100 μM promotes massive nonspecific death of NF1+/− and Nf1−/− cells and was excluded from analysis. Nitroxoline at concentrations ranging from 33 μM to 1 μM reduced proliferative activity of the NF1−/− cells (except clone 3) while proliferation of NF1+/− cells was not affected (FIG. 3).

At higher concentrations, 33 μM: Ratio MIFmean=0,39+/−0,11 and 10 μM: Ratio MIFmean=0,5+/−0,18, proliferation of NF1−/− cells is more affected compared to lower concentrations 3 μM: Ratio MIFmean=0,60+/−0,22 and 1 μM: Ratio MIFmean=0,7+/−0,26 suggesting that the proliferation rate of NF1−/− cells is drug dose dependent and that 33 μM and 10 μM concentrations in this assay present the best efficacy/toxicity ratio.

### Cell Death

At 72 h of culture, cells were labelled for quantification of cell death using DAPI (4′,6-diamidino-2-phenylindole). Labelled cells were analyzed by cytometry with LSR Fortessa X20 (BD Biosciences).

$$Ratio : MIF ( DMSO ) / MIF ( treatment ) =$$

$$If ratio < 1 : decrease in proliferation compared to DMSO$$

$$If ratio > 1 : increase in proliferation compared to DMSO$$

Nitroxoline at 100 μM promotes massive cell death of NF1−/− and NF1+/− cells probably due to its toxic effect and was excluded for the analysis. Interestingly, Nitroxoline at 33 μM promoted cell death of NF1−/− (Ratio dead cells mean=8,86+/−0,66) without affecting NF1+/− cells. Lower concentrations of nitroxoline slightly promote the death of mutant cells without affecting control conditions (10 μM: Ratio dead cells mean=2,04+/−0,63).

### Example 2—In Vivo Drug Testing in Nf1-KO Mice

### Animals

This study uses the same mouse model that was used for in vitro testing, the Prss56Cre/+, R26tdTom/+, NF1f/l mouse (Radomska et a., 2019). This study is a standard experimental design in the field with endpoints being terminal and not longitudinal throughout study.

Prss56Cre/+, R26tdTom/+, NF19/fl herein referred to as Nf1-KO mice develop cNFs after one year of age. However, cNF development occurs at an earlier age after skin injury induced by mice fighting, which is normal mouse behaviour. In this experiment, at least five six-week old Nf1-KO male mice were placed in a cage together, once the mice had sustained a level of skin injury via fighting they were removed from the cage and monitored. At approximately three months of age the mice that had sustained fighting injuries developed several cNFs. As a fluorescent reporter gene is turned on in cells that have Nf1 deleted, cNF development could be monitored via fluorescent imaging of the skin using an epifluorescent microscope (Leica MZ75). After 1-2 months mice that had established, mature cNFs were then enrolled into a drug treatment study. The mice were either treated with vehicle via oral gavage (20% DMSO in 80% corn oil) or with 120 mg/kg nitroxoline via oral gavage once a day (5 days on, 2 days off) for one month.

### Ex Vivo Analysis

Mice were sacrificed after one month of treatment. Epifluorescent imaging was used to guide dissection of skin areas that had cNFs present. cNFs were dissected by punch biopsy (5 mm) and fixed in 4% PFA overnight, then cryoprotected in 30% sucrose. Finally, samples were embedded in gelatin/sucrose (15%/7.5%). cNFs were cryosectioned (14 um) and fluorescent Tom+ cells were imaged using a fluorescent microscope (Leica M165FC).

### Results

### In Vivo Efficacy

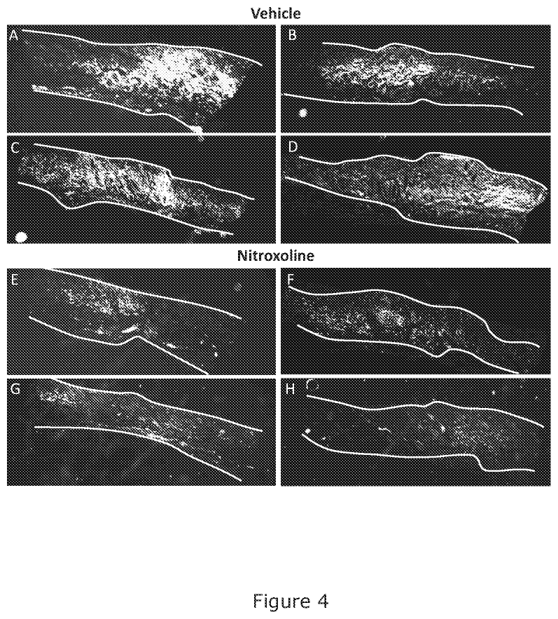

Schwann cells that are Nf1-null also express the fluorescent reporter tomato (Tom+). cNFs are derived from Nf1-null SCs which are Tom+, therefore fluorescence can be used to identify tumours. Using low power (5×) magnification the inventors were able to visualise fluorescent cNF tumour cells in the mouse skin (FIG. 4). It can be seen in these images that the tumours in the nitroxoline treated mice (E-H) are smaller than the tumours in the vehicle treated mice (A-D) after one month of treatment. Therefore, these images indicate that nitroxoline treatment at 120 mg/kg once daily reduces the overall abundance of cNF tumour cells in the skin.

### CONCLUSIONS

Nitroxoline (10 μM to 33 μM) inhibits proliferation and induces cell death in tumor cells isolated from cutaneous neurofibromas derived from the NF1−/− mouse model. The effect was not observed in cells from NF1+/− mice which suggests the nitroxoline effect is dependent on complete deletion of Neurofibromin. Further to the in vitro findings, the inventors also show that nitroxoline is able to reduce (in vivo) the number of tumour cells in cNFs that develop in mice. It is thus expected that nitroxoline will reduce, treat and prevent cutaneous neurofibromas.

### REFERENCES

Radomska K J, Coulpier F, Gresset A, Schmitt A, Debbiche A, Lemoine S, Wolkenstein P, Vallat J M, Charnay P, Topilko P. Cellular Origin, Tumor Progression, and Pathogenic Mechanisms of Cutaneous Neurofibromas Revealed by Mice with Nf1 Knockout in Boundary Cap Cells. Cancer Discov. 2019 January; 9(1):130-147.

1. A method of treating cutaneous neurofibroma, comprising:
administering a composition comprising nitroxoline, or a pharmaceutically acceptable salt thereof, to a subject in need thereof.

2. (canceled)

3. The method of 1 wherein the subject has neurofibromatosis type I.

4. The method of claim 1, wherein the subject is human.

5. The method of claim 1, wherein the composition comprises 30 mg to 600 mg of nitroxoline.

6. The method of claim 1, wherein administration is by a dose two times per day.

7. The method of claim 6, wherein each dose comprises 45 mg to 900 mg of nitroxoline.

8. The method of claim 1, wherein administration is by a dose three times per day.

9. The method of claim 8, wherein each dose comprises 30 mg to 600 mg of nitroxoline.

10. The method of claim 1, wherein administration is by a dose four times per day.

11. The method of claim 10, wherein each dose comprises 15 mg to 500 mg of nitroxoline.

12. The method of claim 1, wherein the composition is administered orally or intravenously.

13. The method of claim 1, wherein the composition is administered by parenteral, transdermal, sublingual, rectal or inhaled administration.

14. The method of claim 1, wherein the nitroxoline, or pharmaceutically acceptable salt thereof, is the only active agent in the composition.

15. The method of claim 1, wherein the composition further comprises selumetinib, or a pharmaceutically acceptable salt thereof.

16. The method of claim 1, wherein:
the treatment comprises administering a second composition comprising selumetinib, or a pharmaceutically acceptable salt thereof; and
the composition comprising nitroxoline, or a pharmaceutically acceptable salt thereof, and the second composition are administered to the subject simultaneously, separately or sequentially.

17. The method of claim 15, wherein the composition comprises 1 mg to 75 mg of selumetinib.

18-21. (canceled)

22. The method of claim 16, wherein the second composition comprises 1 mg to 75 mg of selumetinib.
